(19) United States
(12) Plant Patent Application Publication
(10) Pub. No.: US 2006/0248620 P1
(43) Pub. Date: Nov. 2, 2006

(54) Little Honey: a Grevillea cultivar

(76) Inventors: Denis Eric Cox, Queensland (AU); Janice Evelyn Glazebrook, Queensland (AU)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/118,736

(22) Filed: Apr. 29, 2005

(51) Int. Cl. A01H 5/00

(52) U.S. Cl. PLT/226

(57) ABSTRACT

A new and distinct cultivar of Grevillea, named Grevillea ‘Little Honey,’ is provided.

### FIELD OF THE INVENTION

A new and distinct cultivar of Grevillea, named Grevillea ‘Little Honey,’ is provided.

Grevilla shrubs and trees come from Australia and are grown for their beautiful fern-like foliage and small tubular flowers. They can grow outside in mild climates such as California. The flowers of these plants grow in clusters at the ends of the branches. They are about half of an inch long and typically come in reds, yellows, or pinks.

The plant of the subject invention is characterized by its orange flowers, yellow styles, and fine leaves with creamy-silvery undersides.

Grevillea ‘Little Honey’ looks fantastic in a pot, makes an ideal landscaping plant and is suitable for all size gardens from the biggest to the smallest.

### BACKGROUND OF INVENTION

A new seedling arose in a Burpengary, QLD garden bed of Dennis Cox and Janice Glazebrook in 1990. The seedling came up in this garden near a plant of G. Honey Gem and G. Banksii white dwarf. They are the suspected parents. The seed parent G. Honey Gem is characterized by orange colored flower, orange style, broader leaves with yellowy undersides, and larger plant height (6 m). The pollen parent is characterized by cream colored flower, cream style, and more lobed silvery leaves. The new plant was grown by cuttings for 3 generations. This plant was selected because of its smaller growth form and profuse flowering.

### BRIEF DESCRIPTION OF THE PHOTOGRAPHS

FIG. 1 Photograph comparing three different types of Grevillea; ‘Honey Gem’, ‘Star Fire’, and ‘Little Honey’.

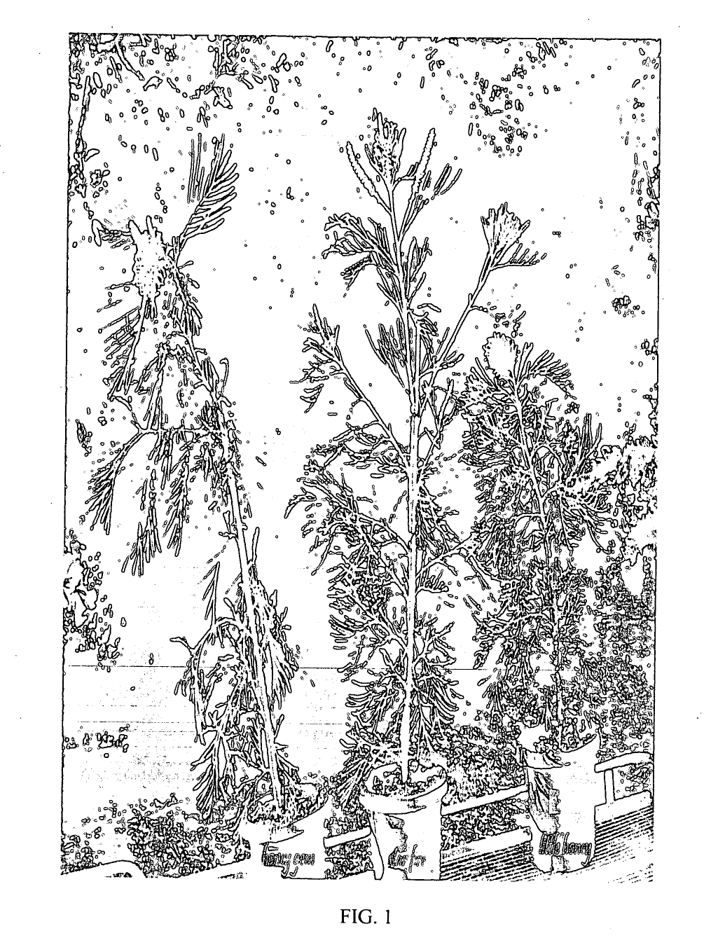

FIG. 2 Photograph comparing three different types of Grevillea; ‘Honey Gem’, ‘Star Fire’, and ‘Little Honey’ This photograph is a view of the typical leaves of each cultivar.

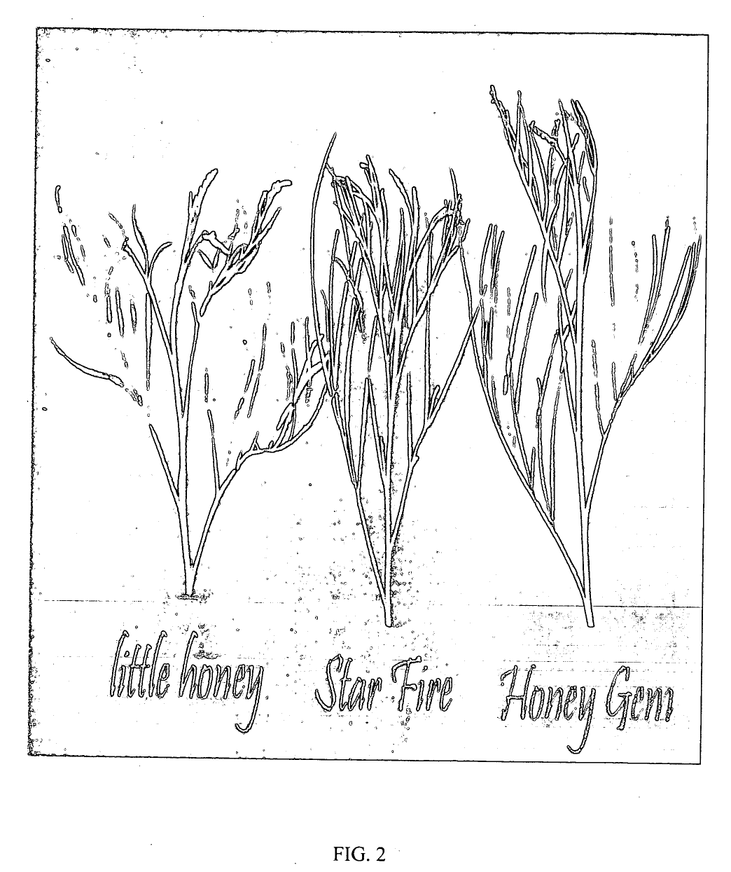

FIG. 3 Close up photograph showing the typical leaves of ‘Honey Gem’.

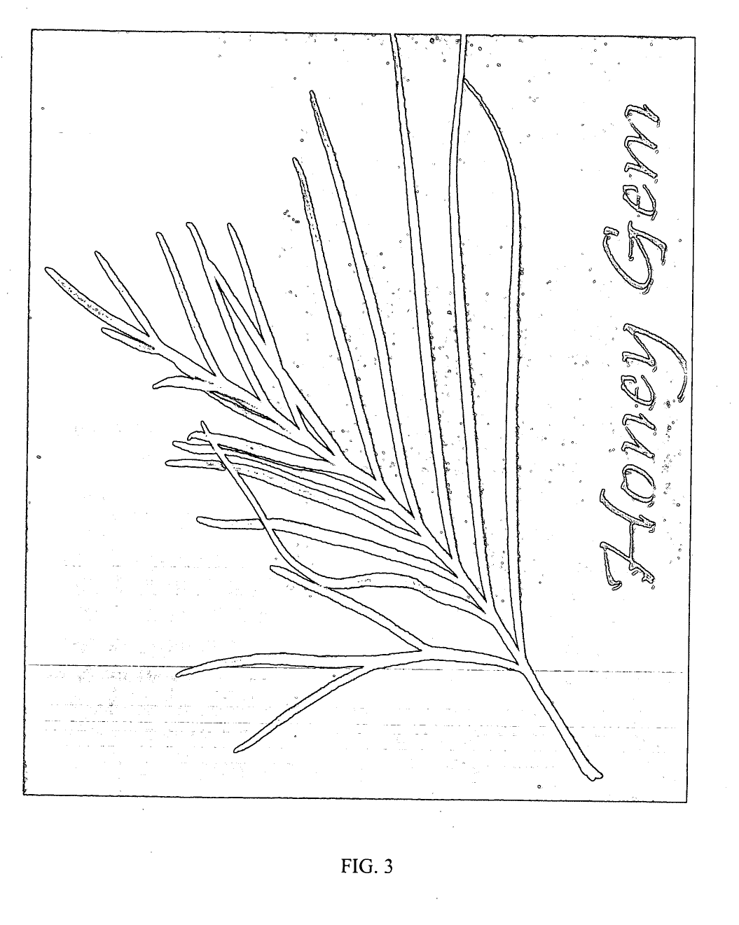

FIG. 4 Close up photograph showing the typical leaves of ‘Star Fire’.

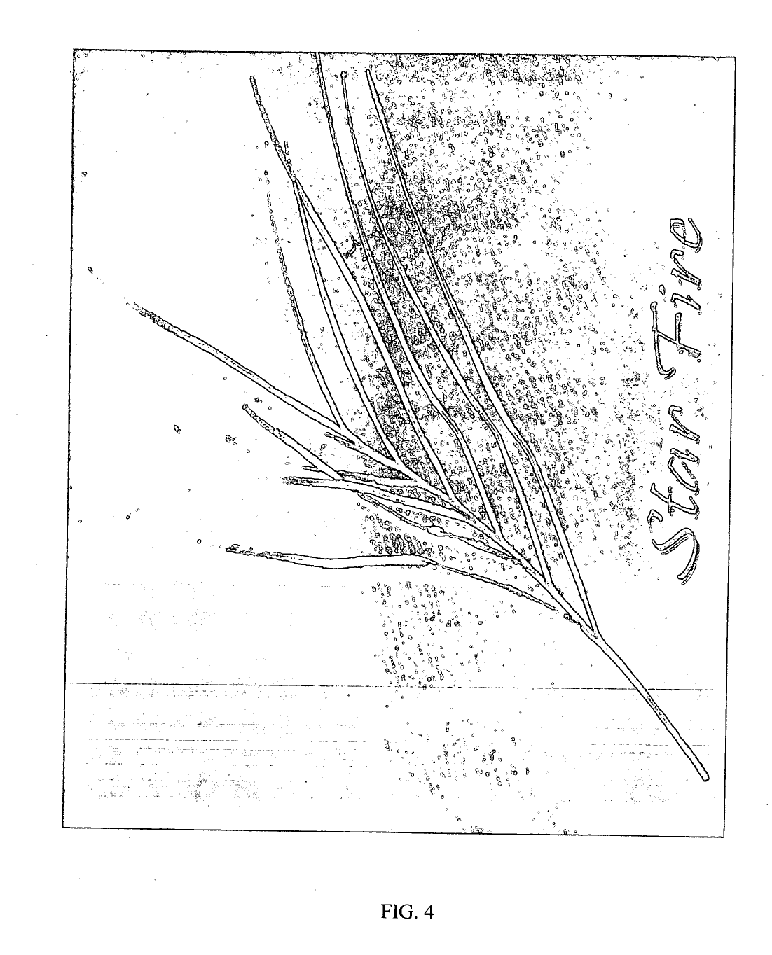

FIG. 5 Close up photograph showing the typical leaves of ‘Little Honey’.

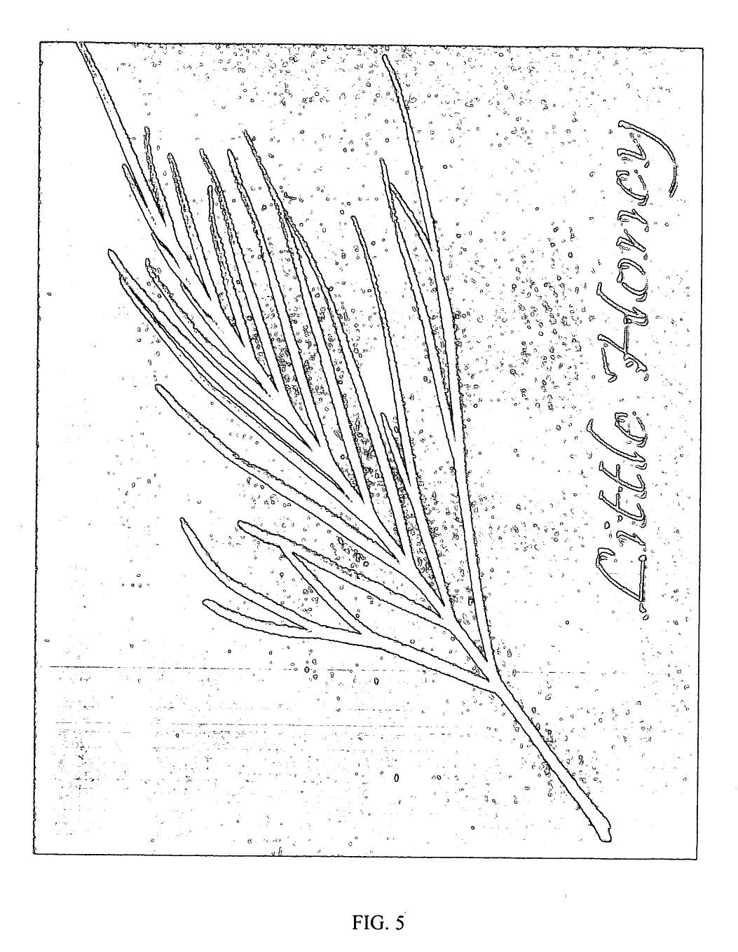

FIG. 6 Photograph comparing three different types of Grevillea; ‘Honey Gem’, ‘Star Fire’, and ‘Little Honey’. This photograph is a view of the typical blooms of each cultivar.

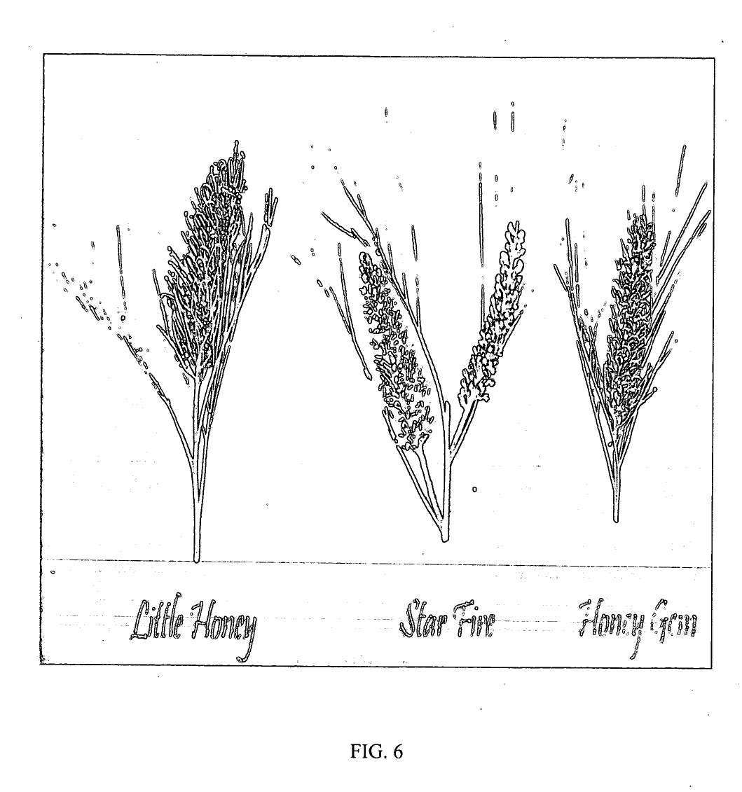

FIG. 7 Close up photograph of the typical blooms of ‘Little Honey’.

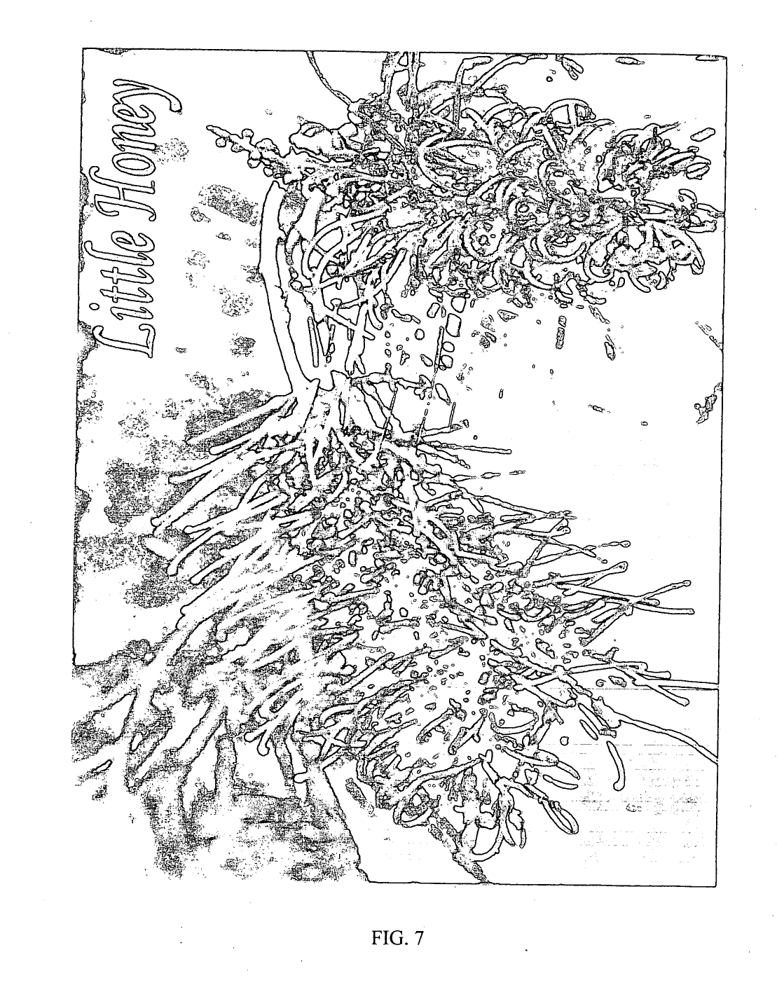

### DETAILED DESCRIPTION OF THE NEW VARIETY

This plant is advantageous in at least the following aspects:
Its attractive foliage
Its unique pale flowers
Nicely-shaped shrub
Profuse flowering

### DETAILED BOTANICAL DESCRIPTION

The following observations, measurements and comparisons describe the cultivar grown in Burpengary, QLD in open conditions on a gravel bed. Tube stock of each variety was planted into 200 mm pots of standard bark potting mix.

The following description uses color references to The Royal Horticultural Society Color Chart, except where general terms of ordinary dictionary significance are used.
Botanical classification: Grevillea hybrid cultivar Grevillea ‘Little Honey’.
Parentage: Seedling.
Propagation: ‘Little Honey’ needs to be reproduced from a cutting to maintain the unique characteristics such as flower, foliage, shape and size.
Plant description:
   Plant size.—average height attained about 2 m: medium; habit: sparse to medium bushy shrub.
   Leaf.—mean length: 222.8 mm; mean width: 160.0 mm; mean lobe width: 2.50 mm; degree of division: deep; variegation: absent; color of lower side of leaf: green (RHS 139A); color of upper side of leaf: greyed green (RHS 190A).
   Inflorescence.—form: cylindrical; position: terminal or at end of lateral stems; length: long; attitude: semi-erect; density of florets: dense.
   Perianth.—color: yellow (RHS 6B); length; short; pubescence: present; overall degree of pubescence: medium.
   Style.—color: orange (RHS 24D); length: short.
   Pollen presenter.—color: yellow (RHS 6B).
   Ovary.—pubescence: present; degree of pubescence: strong.

We claim:

1. A new and distinct cultivar of Grevillea, named Grevillea ‘Little Honey’ as illustrated and described.
